(19) United States
(12) Patent Application Publication
(10) Pub. No.: US 2022/0031630 A1
(43) Pub. Date: Feb. 3, 2022

(54) NANOPARTICLE FORMULATIONS AND METHODS OF THEIR USE

(71) Applicants: The Brigham and Women's Hospital, Inc., Boston, MA (US); Massachusetts Institute of Technology, Cambridge, MA (US); Children's Medical Center Corporation, Boston, MA (US)

(72) Inventors: Jeffrey M. KARP, Boston, MA (US); Wen LI, Boston, MA (US); Nitin JOSHI, Boston, MA (US); Robert S. LANGER, Boston, MA (US); Rebekah MANNIX, Boston, MA (US); Jianhua QIU, Boston, MA (US); Sezin ADAY, Boston, MA (US)

(21) Appl. No.: 17/275,266

(22) PCT Filed: Sep. 13, 2019
(86) PCT No.: PCT/US2019/051108

§ 371 Date: Mar. 11, 2021

Related U.S. Application Data
(60) Provisional application No. 62/733,127, filed on Sep. 19, 2018.
(60) Provisional application No. 62/730,829, filed on Sep. 13, 2018.

(51) Int. Cl. A61K 9/51; A61K 9/00; A61K 38/06; A61K 31/7088

(52) U.S. Cl. CPC A61K 9/5153; A61K 9/0085; A61K 31/7088; A61K 38/063; A61K 9/5123

(57) ABSTRACT

Disclosed are pharmaceutical compositions formulated for delivery to the brain of a subject. The compositions include a plurality of nanoparticles (NPs) containing a brain therapeutic agent, poly(lactic-co-glycolic acid) (PLGA), and a pharmaceutically acceptable excipient selected from the group consisting of a surfactant, peptide, and combinations thereof. Also disclosed are methods of their use.

### CROSS-REFERENCE TO RELATED APPLICATIONS

This application claims the benefit of U.S. Provisional Application No. 62/730,829, filed Sep. 13, 2018, and U.S. Provisional Application No. 62/733,127, filed Sep. 19, 2018, each of which is incorporated by reference herein in its entirety.

### STATEMENT REGARDING FEDERALLY SPONSORED RESEARCH OR DEVELOPMENT

This invention was made with Government support under Grant Nos. HL095722 and DE013023 awarded by the National Institutes of Health. The Government has certain rights in the invention.

### FIELD OF THE INVENTION

The invention relates to nanoparticulate formulations, e.g., formulations capable of delivering brain therapeutic agents across the blood brain barrier.

### BACKGROUND

A blood brain barrier (BBB) separates the circulating blood from the brain and extracellular fluid in the nervous system. It is formed by endothelial cells of the capillary walls and presents a formidable challenge for drug delivery, as numerous therapeutic agents fail to cross the blood brain barrier.

Nanoparticulate formulations are one of the approaches for pharmaceutical drug delivery. Such formulations, however, may suffer from poor therapeutic agent encapsulation efficiency and/or poor therapeutic agent release efficiency.

There is a need for new formulation approaches for delivering therapeutic agents across the blood brain barrier.

### SUMMARY OF THE INVENTION

In general, the invention provides a pharmaceutical composition formulated for delivery to a subject (e.g., to the brain of the subject). The composition includes a plurality of nanoparticles (NPs) including a cargo molecule (e.g., a brain therapeutic agent), a polymer (e.g., poly(lactic-co-glycolic acid) (PLGA)), and a pharmaceutically acceptable excipient (e.g., a surfactant, peptide, or a combination thereof). Preferably, the cargo molecule is a brain therapeutic agent. Preferably, the polymer is PLGA. As is disclosed herein, the described nanoparticle formulations advantageously deliver their cargo across breached or intact blood brain barrier.

In some embodiments, the brain therapeutic agent treats a functional disorder. In certain embodiments, the brain therapeutic agent treats a physical disorder (e.g., a traumatic brain injury).

In further embodiments, the pharmaceutical composition includes a surfactant (e.g., a polysorbate, polyethylene glycol, or poloxamer). Preferably, the surfactant is a polysorbate (e.g., polysorbate 80). In yet further embodiments, the pharmaceutical composition includes 0.001-0.2% (w/v) of the surfactant. In still further embodiments, the pharmaceutical composition includes a peptide (e.g., glutathione, transferrin, or a combination thereof). Preferably, the peptide is glutathione. In other embodiments, the pharmaceutical composition includes 0.05-0.5% (w/v) of the peptide.

In yet other embodiments, the NPs have an average hydrodynamic diameter of 40-150 nm (e.g., 50-150 nm or 40-100 nm), as measured by Dynamic Light Scattering. In still other embodiments, the NPs have an average hydrodynamic diameter of 55-95 nm, as measured by Dynamic Light Scattering.

In some embodiments, the average molecular weight of the NPs is 7-31 kDa. In certain embodiments, the average diameter of the NPs is 40-70 nm, as measured by transmission electron microscopy (TEM). In particular embodiments, the brain therapeutic agent is a nucleic acid.

In further embodiments, the nucleic acid is a plasmid, siRNA, shRNA, miRNA, an antisense oligonucleotide, gRNA (e.g., sgRNA), an aptamer, or a combination thereof. Preferably, the nucleic acid is siRNA or antisense oligonucleotide.

In yet further embodiments, the brain therapeutic agent is selected from the group consisting of anti-inflammatory drugs, steroids, antibiotics, immunosuppressants, chemotherapeutics, sensitizing agents, antibodies, antibody fragments, proteins, peptides, growth factors, cytokines, cells, stem cells, vitamins, and combinations thereof.

In still further embodiments, the pharmaceutical composition includes a solvent (e.g., water, saline, or phosphate-buffered saline).

In other embodiments, the pharmaceutical composition includes 0.5-50 mg/mL of a polymer (e.g., PLGA).

In another aspect, the invention provides a method of delivering a cargo (e.g., a brain therapeutic agent) to a subject in need thereof by administering to the subject an effective amount of the pharmaceutical composition disclosed herein.

In some embodiments, the method treats a functional disorder in the subject. In certain embodiments, the functional disorder is a mental disorder. In particular embodiments, the functional disorder is a physical disorder (e.g., a traumatic brain injury). In further embodiments, the functional disorder is a traumatic brain injury and the brain therapeutic agent is a nucleic acid. In yet further embodiments, the pharmaceutical composition is administered intravenously.

The invention is further described by the following enumerated items.

1. A pharmaceutical composition formulated for delivery to the brain of a subject, the composition including a plurality of nanoparticles (NPs) including a brain therapeutic agent, poly(lactic-co-glycolic acid) (PLGA), and a pharmaceutically acceptable excipient selected from the group consisting of a surfactant, peptide, and combinations thereof.

2. The pharmaceutical composition of item 1, wherein the brain therapeutic agent treats a functional disorder.

3. The pharmaceutical composition of item 1, wherein the brain therapeutic agent treats a physical disorder.

4. The pharmaceutical composition of item 3, wherein the physical disorder is a traumatic brain injury.

5. The pharmaceutical composition of any one of items 1 to 4, wherein the pharmaceutical composition includes a surfactant.

6. The pharmaceutical composition of any one of items 1 to 5, wherein the surfactant is a polysorbate, polyethylene glycol, or poloxamer.

7. The pharmaceutical composition of item 6, wherein the surfactant is a polysorbate.

8. The pharmaceutical composition of item 7, wherein the polysorbate is polysorbate 80.

9. The pharmaceutical composition of any one of items 1 to 8, wherein the pharmaceutical composition includes 0.001-0.2% (w/v) (e.g., 0.1-0.2% (w/v)) of the surfactant.

10. The pharmaceutical composition of any one of items 1 to 9, wherein the pharmaceutical composition includes a peptide.

11. The pharmaceutical composition of any one of items 1 to 10, wherein the peptide is glutathione, transferrin, or a combination thereof.

12. The pharmaceutical composition of item 11, wherein the peptide is glutathione.

13. The pharmaceutical composition of any one of items 1 to 12, wherein the pharmaceutical composition includes 0.05-0.5% (w/v) (e.g., 0.1-0.2% (w/v)) of the peptide.

14. The pharmaceutical composition of any one of items 1 to 13, wherein the NPs have an average hydrodynamic diameter of 40-150 nm (e.g., 50-150 nm or 40-100 nm), as measured by Dynamic Light Scattering.

15. The pharmaceutical composition of any one of items 1 to 13, wherein the NPs have an average hydrodynamic diameter of 55-95 nm, as measured by Dynamic Light Scattering.

16. The pharmaceutical composition of any one of items 1 to 15, wherein the average molecular weight of the NPs is 7-31 kDa.

17. The pharmaceutical composition of any one of items 1 to 16, wherein the average diameter of the NPs is 40-70 nm, as measured by transmission electron microscopy (TEM).

18. The pharmaceutical composition of any one of items 1 to 17, wherein the brain therapeutic agent is a nucleic acid.

19. The pharmaceutical composition of item 18, wherein the nucleic acid is a plasmid, siRNA, shRNA, miRNA, antisense oligonucleotide, gRNA (e.g., sgRNA), aptamer, or a combination thereof.

20. The pharmaceutical composition of item 19, wherein the nucleic acid is siRNA or antisense oligonucleotide.

21. The pharmaceutical composition of any one of items 1 to 17, wherein the brain therapeutic agent is selected from the group consisting of anti-inflammatory drugs, steroids, antibiotics, immunosuppressants, chemotherapeutics, sensitizing agents, antibodies, antibody fragments, proteins, peptides, growth factors, cytokines, cells, stem cells, vitamins, and combinations thereof.

22. The pharmaceutical composition of any one of items 1 to 21, wherein the pharmaceutical composition includes a solvent.

23. The pharmaceutical composition of item 22, wherein the solvent is water, saline, or phosphate-buffered saline.

24. The pharmaceutical composition of any one of items 1 to 23, wherein the pharmaceutical composition includes 0.5-50 mg/mL of PLGA.

25. A method of delivering a brain therapeutic agent to a subject in need thereof, the method including administering to the subject an effective amount of the pharmaceutical composition of any one of items 1-24.

26. The method of item 25, wherein the method treats a functional disorder in the subject,

27. The method of item 26, wherein the functional disorder is a mental disorder.

28. The method of item 26, wherein the functional disorder is a physical disorder.

29. The method of item 28, wherein the physical disorder is a traumatic brain injury.

30. The method of item 26, wherein the functional disorder is a traumatic brain injury and the brain therapeutic agent is a nucleic acid.

31. The method of any one of items 25-30, wherein the pharmaceutical composition is administered intravenously.

32. A pharmaceutical composition including a plurality of nanoparticles (NPs) including a cargo molecule, a hydrophobic polymer, and a pharmaceutically acceptable excipient selected from the group consisting of a surfactant, peptide, and combinations thereof.

33. The pharmaceutical composition of any one of items 32, wherein the pharmaceutical composition includes a surfactant.

34. The pharmaceutical composition of any one of items 32 to 33, wherein the surfactant is a polysorbate, polyethylene glycol, or poloxamer.

35. The pharmaceutical composition of item 34, wherein the surfactant is a polysorbate.

36. The pharmaceutical composition of item 35, wherein the polysorbate is polysorbate 80.

37. The pharmaceutical composition of any one of items 32 to 36, wherein the pharmaceutical composition includes 0.001-0.2% (w/v) (e.g., 0.1-0.2% (w/v)) of the surfactant.

38. The pharmaceutical composition of any one of items 32 to 37, wherein the pharmaceutical composition includes a peptide.

39. The pharmaceutical composition of any one of items 32 to 38, wherein the peptide is glutathione, transferrin, or a combination thereof.

40. The pharmaceutical composition of item 39, wherein the peptide is glutathione.

41. The pharmaceutical composition of any one of items 32 to 40, wherein the pharmaceutical composition includes 0.05-0.5% (w/v) (e.g., 0.1-0.2% (w/v)) of the peptide.

42. The pharmaceutical composition of any one of items 32 to 41, wherein the NPs have an average hydrodynamic diameter of 40-150 nm (e.g., 50-150 nm or 40-100 nm), as measured by Dynamic Light Scattering.

43. The pharmaceutical composition of any one of items 32 to 42, wherein the NPs have an average hydrodynamic diameter of 55-95 nm, as measured by Dynamic Light Scattering.

44. The pharmaceutical composition of any one of items 32 to 43, wherein the average molecular weight of the NPs is 7-31 kDa.

45. The pharmaceutical composition of any one of items 32 to 44, wherein the average diameter of the NPs is 40-70 nm, as measured by transmission electron microscopy (TEM).

46. The pharmaceutical composition of any one of items 32 to 45, wherein the cargo molecule is a nucleic acid, an anti-inflammatory drug, a steroid, an antibiotic, an immunosuppressant, a chemotherapeutic, a sensitizing agent, an antibody or fragment thereof, a protein or peptide, a growth factor, a cytokine, a cell (e.g., a stem cell), a vitamin, or any combination of the foregoing cargoes.

47. The pharmaceutical composition of any one of items 32 to 46, wherein the pharmaceutical composition includes a solvent.

48. The pharmaceutical composition of item 47, wherein the solvent is water, saline, or phosphate-buffered saline.

49. The pharmaceutical composition of any one of items 32 to 48, wherein the hydrophobic polymer is a biodegradable hydrophobic polymer.

50. The pharmaceutical composition of any one of items 32 to 48, wherein the hydrophobic polymer is poly(lactic-co-glycolic acid) (PLGA), poly(caprolactone) (PCL), poly(lactic acid) (PLA), poly(glycolic acid) (PGA), poly(caprolactone)-co-poly(lactic acid) (PCLLA), poly(L-lactic acid) (PLLA), or poly(glycerol sebacate) acrylate (PGSA).

51. The pharmaceutical composition of any one of items 32 to 50, wherein the hydrophobic polymer is PLGA.

52. The pharmaceutical composition of any one of items 32 to 51, wherein the pharmaceutical composition includes 0.5-50 mg/mL of the hydrophobic polymer.

### Definitions

The term “about,” as used herein, represents a value that is in the range of ±10% of the value that follows the term “about.”

The term “nanoparticle,” as used herein, refers to particle having an average hydrodynamic diameter of 1 to 1000 nm, as measured by Dynamic Light Scattering. Preferably, nanoparticles have an average hydrodynamic diameter of about 40-150 nm (e.g., 50-150 nm or 40-100 nm). More preferably, nanoparticles have an average hydrodynamic diameter of about 40-100 nm.

The term “pharmaceutically acceptable,” as used herein, refers to those compounds, materials, compositions, and/or dosage forms, which are suitable for contact with the tissues of an individual (e.g., a human), without excessive toxicity, irritation, allergic response and/or other complications commensurate with a reasonable benefit/risk ratio.

The term “pharmaceutical composition,” as used herein, represents a composition containing one or more cargoes such as therapeutic agents described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Preferably, nanoparticles are formulated for intravenous administration.

The term “polysorbate,” as used herein, refers to oleate esters of sorbitol and its anhydrides, typically copolymerized with ethylene oxide. A preferred polysorbate is polysorbate 80 (poly(ethylene oxide) (80) sorbitan monolaurate).

The term “subject,” as used herein, represents a human or non-human animal (e.g., a mammal) that is suffering from a disease, disorder, or condition affecting the tissues of the subject (e.g., a disease, disorder, or condition which is protected from blood by a blood brain barrier (intact blood brain barrier or a breached blood brain barrier)), as determined by a qualified professional (e.g., a doctor or a nurse practitioner) with or without known laboratory test(s) or sample(s) from the subject.

“Treatment” and “treating,” as used herein, refer to the medical management of a subject with the intent to improve, ameliorate, stabilize, prevent or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

### BRIEF DESCRIPTION OF THE DRAWINGS

FIGS. 1A-1E show the preparation and characterization of different surface chemistries-coated siRNA-loaded NPs.

FIG. 1A shows the outline for the preparation of siRNA-loaded PLGA NPs by a modified nanoprecipitation method.

FIG. 1B shows photographs of transmission electron microscopy (TEM) images of siRNA-loaded NPs with different surface coatings. Scale bar=100 nm.

FIG. 1C the size of siRNA-loaded NPs with different surface coatings analyzed by dynamic light scattering via a Zetasizer.

FIG. 1D shows the zeta potential of siRNA-loaded NPs with different surface coatings analyzed by dynamic light scattering via a Zetasizer.

FIG. 1E shows the encapsulation efficiency of siRNA in different NPs.

FIGS. 2A-2D show the cellular uptake, endosome escape, and gene silencing ability of different nanoplatforms.

FIG. 2A shows confocal laser scanning microscope (CLSM) images of Neuro 2A cells incubated with free siRNA, or different siRNA-loaded NPs.

FIG. 2B shows the lysosome escape of siRNA by CLSM images. Neuro 2A cells were incubated with free siRNA, or different siRNA-loaded NPs.

FIG. 2C shows the viability of Neuro 2A cells after incubation with free siRNA, lipofectamine 2000+siRNA, or different siRNA-loaded NPs.

FIG. 2D shows the luciferase expression in Neuro 2A cells. The luciferase-expressing Neuro 2A cells were treated with free luciferase siRNA, lipofectamine 2000+luciferase siRNA, or luciferase siRNA-loaded different NPs.

FIG. 3A shows the in vivo brain accumulation efficiency of different surface coating-modified siRNA NPs. IVIS images of excised mice brains after systematic injection of different siRNA-loaded NPs into mice.

FIG. 3B shows quantitative analysis of siRNA fluorescent intensity in excised mice brains.

FIGS. 4A and 4B show size and zeta potential of siRNA-loaded NPs with low, medium, or high coating densities of poly80 or GSH.

FIG. 4C demonstrates encapsulation efficiency of siRNA in different PLGA NPs.

FIG. 4D is a series of fluorescence microscope images of Neuro 2A cells incubated with different siRNA-loaded NPs.

FIG. 4E demonstrates the viability of Neuro 2A cells after incubation with different siRNA-loaded NPs.

FIG. 4F demonstrates the luciferase expression in Neuro 2A cells. The luciferase-expressing Neuro 2A cells were treated with different luciferase siRNA-loaded NPs.

FIG. 5A is a series of IVIS images of excised mice brains at 4 hours after systematic injection of different siRNA-loaded NPs into mice.

FIG. 5B shows a quantitative analysis of siRNA fluorescent intensity in excised mice brains at 4 hours after systematic injection of different siRNA-loaded NPs into mice.

FIG. 6A is a series of IVIS images of excised mice brains at 4 hours after systematic injection of different siRNA-loaded PS 80 NPs into healthy mice.

FIG. 6B is a series of IVIS images of brain coronal section from mice.

FIG. 7A is a schematic illustration of the isolation of primary neural cells from mouse embryos, and the bright-field images of primary cells after 1 week in culture.

FIG. 7B is a western blot analysis and quantification of Tau expression in primary neural cells after treatment of PBS (1), free Tau siRNA (2), Tau siRNA-loaded PEG NPs (3), Tau siRNA-loaded PS 80 (H) NPs (4), and random siRNA-loaded PS 80 (H) NPs (5).

FIG. 7C is a western blot analysis and quantification of Tau expression in primary neural cells after treatment of Tau siRNA-loaded PS 80 (H) NPs at different siRNA dose.

FIG. 7D is a series of immunofluorescence images of primary cells after treatment of PBS, Tau siRNA, Tau siRNA-loaded PEG NPs, and different dose of Tau siRNA-loaded PS 80 (H) NPs.

FIG. 8A illustrates the weight drop-induced TBI model.

FIG. 8B shows BBB permeability of mice before or at different time points following TBI, as assessed by Evans blue penetration assay.

FIG. 8C is a series of IVIS images of excised mice brains 4 hours after injection of free siRNA, siRNA-loaded PEG NPs, or siRNA-loaded PS 80 (H) NPs. The solutions were injected into TBI mice immediately after injury or 2 weeks after injury.

FIG. 9A is a schematic representation showing experimental outline for studying Tau silencing efficacy in vivo. Injury was induced on day 0 followed by a tail vein injection of free Tau siRNA, siRNA-loaded NPs on day 0 and day 1. Brains were harvested on day 4.

FIG. 9B shows the western blot analysis of Tau expression in brain in naïve vs TBI mice treated with free Tau siRNA or Tau siRNA-loaded PS 80 NPs.

FIG. 9C shows the experimental outline for studying Tau silencing efficacy when different formulations were administrated at 2 weeks following TBI.

FIG. 9D shows the western blot analysis of Tau expression in brain in naïve vs TBI mice treated with free Tau siRNA, Tau siRNA or control siRNA-loaded NPs at 2 weeks after TBI.

FIG. 9E is a series of immunochemical microphotographs of Tau expression in brain tissue before and after treatment with free Tau siRNA or Tau siRNA-loaded PS 80 NPs. The treatments were performed either on day 0 and day 1 or on day 14 and day 15.

### DETAILED DESCRIPTION

The invention provides pharmaceutical compositions formulated for delivery to a subject (e.g., the brain of a subject). The pharmaceutical compositions include a plurality of nanoparticles (NPs) containing a cargo molecule (e.g., brain therapeutic agent), a polymer (e.g., a hydrophobic polymer, e.g., a biodegradable hydrophobic polymer such as poly(lactic-co-glycolic acid) (PLGA)), and a pharmaceutically acceptable excipient, e.g., a surfactant, peptide, gelator, small molecule, or a combination thereof. Preferably, the pharmaceutically acceptable excipient is a surfactant, peptide, or combination thereof. More preferably, the excipient is a polysorbate, glutathione, or a combination thereof. Preferably, the polymer is a hydrophobic polymer. More preferably, the polymer is a biodegradable hydrophobic polymer (e.g., PLGA). Typically, a pharmaceutical composition disclosed herein is an aqueous composition.

Advantageously, pharmaceutical compositions disclosed herein (e.g., those including preferred excipients) may be used to deliver brain therapeutic agents, even nucleic acids (e.g., siRNA), across the blood brainer barrier. The pharmaceutical compositions of the invention may deliver the brain therapeutic agents across intact blood brainer barrier or across physically breached blood brain barrier. Pharmaceutical compositions disclosed herein (e.g., those including preferred excipients) may advantageously exhibit high therapeutic agent (e.g., nucleic acids, e.g., siRNA) encapsulation efficiency.

Pharmaceutical compositions disclosed herein include nanoparticles (NPs) having an average molecular weight of about 7-31 kDa. In some embodiments, with average hydrodynamic diameter of the NPs is about 40-150 nm (e.g., 50-150 nm or 40-100 nm), as measured by Dynamic Light Scattering. The nanoparticles have an average hydrodynamic diameter that can be, e.g., 40-150 nm (e.g., 50-150 nm or 40-100 nm), but the hydrodynamic diameter can vary widely depending on the required application.

The nanoparticle surface is typically modified with a pharmaceutically acceptable excipient, e.g., a gelator, small molecule, surfactant, peptide, or a combination thereof. Such pharmaceutically acceptable excipient may advantageously improve the cargo therapeutic agent brain accumulation upon delivery across either intact or physically breached blood brain barrier. The pharmaceutically acceptable excipient may be present in the amount up to, e.g., 0.5% (w/v). For example, the pharmaceutical composition disclosed herein may include 0.001-0.5% (w/v) (e.g., 0.001-0.4% (w/v), 0.001-0.3% (w/v), 0.001-0.2% (w/v), 0.01-0.5% (w/v), 0.01-0.4% (w/v), 0.01-0.3% (w/v), 0.01-0.2% (w/v), 0.05-0.5% (w/v), 0.05-0.4% (w/v), 0.05-0.3% (w/v), 0.05-0.2% (w/v), 0.1-0.5% (w/v), 0.1-0.4% (w/v), 0.1-0.3% (w/v), 0.1-0.2% (w/v), 0.2-0.5% (w/v), 0.2-0.4% (w/v), 0.2-0.3% (w/v), 0.3-0.5% (w/v), 0.3-0.4% (w/v), or 0.4-0.5% (w/v)) of the pharmaceutically acceptable excipient (e.g., a surfactant or peptide). The gelator may be, e.g., a prodrug. For example, a pharmaceutically acceptable excipient that is a peptide (e.g., transferrin or GSH) may be present in a pharmaceutical composition disclosed herein in the amount of 0.05-0.3% (w/v) (e.g., 0.05-0.2% (w/v), 0.05-0.1% (w/v), 0.1-0.3% (w/v), 0.1-0.2% (w/v), or 0.2-0.3% (w/v)). Alternatively, a pharmaceutically acceptable excipient that is a peptide (e.g., GSH) may be present in a pharmaceutical composition disclosed herein in the amount of 0.1-0.2% (w/v). In another example, a pharmaceutically acceptable excipient that is a pluronic (e.g., Pluronic-F68 or Pluronic-F127) may be present in a pharmaceutical composition disclosed herein the amount of 0.1-0.5% (w/v) (e.g., 0.1-0.4% (w/v), 0.1-0.3% (w/v), 0.1-0.2% (w/v), 0.2-0.5% (w/v), 0.2-0.4% (w/v), 0.2-0.3% (w/v), 0.3-0.5% (w/v), 0.3-0.4% (w/v), or 0.4-0.5% (w/v)). In yet another example, a pharmaceutically acceptable excipient that is tocopherol-polyethylene glycol succinate (TPGS) may be present in a pharmaceutical composition disclosed herein in the amount of 0.05-0.5% (w/v) (e.g., 0.05-0.4% (w/v), 0.05-0.3% (w/v), 0.05-0.2% (w/v), 0.1-0.5% (w/v), 0.1-0.4% (w/v), 0.1-0.3% (w/v), 0.1-0.2% (w/v), 0.2-0.5% (w/v), 0.2-0.4% (w/v), 0.2-0.3% (w/v), 0.3-0.5% (w/v), 0.3-0.4% (w/v), or 0.4-0.5% (w/v)). In still another example, a pharmaceutically acceptable excipient that is a polysorbate (e.g., polysorbate 80) may be present in a pharmaceutical composition disclosed herein in the amount of 0.1-0.2% (w/v).

The pharmaceutically acceptable excipient used for the nanoparticle surface modification may be, e.g., a pharmaceutically acceptable excipient described herein. Non-limiting examples of such pharmaceutically acceptable excipients include polyvinyl acetate (PVA), pluronic-F127, pluronic-F68, tocopherol-polyethylene glycol succinate (TPGS), 1,2-distearoyl-sn-glycero-3-phosphoethanolamine-N-[amino(polyethylene glycol)] (DSPE-PEG), dioleoyl trimethylammonium propane (DOTAP), lysophosphatidyl choline, tetradecyl maltoside, folic acid, PEG 15 hydroxystearate (Solutol HS15), polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40), polyethylene-polypropylene glycol (poloxamer 124), PEG 8 caprylic/capric glycerides (Labrasol), PEG 300 oleic glycerides (Labrafil M 1944), diethylene glycol monoethyl ether (Transcutol), lauroyl macrogol 32 glycerides (GELUCIRE® 44/14), polyethylene glycol 400 (PEG 400), propylene glycol laurate (Lauroglycol FCC), D-a-Tocopherol polyethylene glycol 1000 succinate (TPGS), polyethylene-polypropylene glycol (poloxamer 188), polyethylene-polypropylene glycol (poloxamer 407), polyvinyl pyrrolidone (e.g., Mw 28-34 kDa, Mw 44-54 kDa (e.g., Kollidon 30), or 1-1.5M kDa (e.g., Kollidon 90)), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, polyethylene-polypropylene glycol (poloxamer 124), polyethylene glycol sorbitan monolaurate (polysorbate 20, TWEEN 20), polyethylene glycol sorbitan monopalmitate (polysorbate 40, TWEEN 40), polyethylene glycol sorbitan monostearate (polysorbate 60, TWEEN 60), polyethylene glycol sorbitan tristearate (polysorbate 65, TWEEN 65), polyethylene glycol sorbitan monooleate (polysorbate 80, TWEEN 80), polyethylene glycol sorbitan trioleate (polysorbate 85, TWEEN 85), polyethylene glycol sorbitan hexaoleate, polyethylene glycol sorbitan tetraoleate, sorbitan monolaurate (Span 20), sorbitan monopalmitate (Span 40), sorbitan monostearate (Span 60), sorbitan tristearate (Span 65), sorbitane monooleate (Span 80), sorbita n trioleate (Span 85), sucrose laurate, sucrose palmitate, sucrose stearate, gamma-cyclodextrin, beta-cyclodextrin (e.g., CAPTISOL) pectin, whey protein, caseinates, quillaia/quillaia saponins, quillaia extract, PEG 8 stearate, PEG 40 stearate, and D-glucosamine.

Further non-limiting examples of such pharmaceutically acceptable excipients include, e.g., Polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40), polyethylene-polypropylene glycol (poloxamer 124), PEG 8 caprylic/capric glycerides (Labrasol), PEG 300 oleic glycerides (Labrafil M 1944), diethylene glycol monoethyl ether (Transcutol), sorbitane monooleate (Span 80), Lauroyl macrogol 32 glycerides (GELUCIRE® 44/14), polyethylene glycol 400 (PEG 400), propylene glycol laurate (Lauroglycol FCC), polysorbate 20 (TWEEN® 20), polysorbate 40 (TWEEN® 40), polysorbate 60 (TWEEN® 60), polysorbate 80 (TWEEN® 80), D-a-Tocopherol polyethylene glycol 1000 succinate (TPGS), polyethylene-polypropylene glycol (poloxamer 188), polyethylene-polypropylene glycol (poloxamer 407), polyvinyl pyrrolidone (Kollidon 30), polyvinyl pyrrolidone (Kollidon 90), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, and combinations thereof.

Yet further non-limiting examples of such pharmaceutically acceptable excipients include, e.g., Lauroyl macrogol 32 glycerides (GELUCIRE® 44/14), polyethylene glycol 400 (PEG 400), propylene glycol laurate (Lauroglycol FCC), polysorbate 20 (TWEEN® 20), polysorbate 40 (TWEEN®40), polysorbate 60 (TWEEN® 60), polysorbate 80 (TWEEN® 80), D-a-Tocopherol polyethylene glycol 1000 succinate (TPGS), polyethylene-polypropylene glycol (poloxamer 188), polyethylene-polypropylene glycol (poloxamer 407), polyvinyl pyrrolidone (Kollidon 30), polyvinyl pyrrolidone (Kollidon 90), Iota Carrageenan, Xanthan gum, locust Bean gum, Kelcogel LT100, acacia gum, guar gum, gamma-Cyclodextrin, Tracacanth gum, hydroxy propyl methylcellulose (HPMC), carboxymethyl cellulose (CMC), microcrystalline cellulose (MCC), lecithin, and combinations thereof.

Non-limiting examples of pharmaceutically acceptable excipients that are peptides include, e.g., Angiopep-2, ApoB (3371-3409), ApoE (159-167)2, COG133, Peptide-22, THR CRT, Leptin30, RVG2 Apamin, MiniAp-4, GSH, G23, TGN, TAT, CPP, Organic cation/carnitine transporters (OCTNs), L-type amino acid transporter (LAT1), glucose transporter (GLUT1), monocarboxylate lactate transporter (MCT1), cationic amino acid transporter (CAT1), choline transporter (ChT), sodium-coupled glucose transporters (SGLTs), low-density lipoproteins (LDLRs), apolipoproteins (Apo) A, B, E, receptor-associated protein (RAP), transferrin (Tf), lactotransferrin, melanotransferrin, TfR antibody (OX26, 8D3, RI7217), leptin, wheat germ agglutinin, diphtheria toxin, insulin, Insulin receptor antibody, and albumin.

Peptides such as albumin, collagen, gelatin and prolamines, for example, zein, and polysaccharides such as alginate can also be used. Some peptides may be provided in NPs as lipid conjugates, e.g., conjugates with DSPE-PEG (e.g., DSPE-PEG-GSH or DSPE-PEG-Tf).

Pharmaceutical compositions disclosed herein include one or more cargo molecules such as a brain therapeutic agent (e.g., a hydrophobic therapeutic agent or a hydrophilic therapeutic agent) encapsulated in the NPs. Non-limiting examples of cargo molecules (e.g., brain therapeutic agents) include, e.g., anti-inflammatory drugs, steroids, antibiotics, immunosuppressants, chemotherapeutics, sensitizing agents, antibodies, antibody fragments, proteins, peptides, growth factors, cytokines, cells (e.g., stem cells), nucleic acids (e.g., siRNA), vitamins, and combinations thereof. The encapsulated cargo molecules (e.g., a brain therapeutic agent) and/or the gelator can be subsequently delivered through hydrolytic or other forms of degradation of the nanoparticle. The nanoparticles can also be used to co-deliver multiple agents, thereby resulting in their synergistic or additive effects.

Further non-limiting examples of cargo molecules (e.g., therapeutic agents such as brain therapeutic agents) include drugs acting at synaptic and neuroeffector junctional sites; general and local analgesics and anesthetics such as opioid analgesics and antagonists; hypnotics and sedatives; drugs for the treatment of psychiatric disorders such as depression, schizophrenia; anti-epileptics and anticonvulsants; Huntington's disease, aging and Alzheimer's disease; neuroprotective agents (such as excitatory amino acid antagonists and neurotropic factors) and neuroregenerative agents; trophic factors such as brain derived neurotrophic factor, ciliary neurotrophic factor, or nerve growth factor; drugs aimed at the treatment of CNS trauma or stroke; and drugs for the treatment of addiction and drug abuse; autacoids and anti-inflammatory drugs; chemotherapeutic agents for parasitic infections and microbial diseases; immunosuppressive agents and anti-cancer drugs; hormones and hormone antagonists; heavy metals and heavy metal antagonists; antagonists for non-metallic toxic agents; cytostatic agents for the treatment of cancer; diagnostic substances for use in nuclear medicine, and radiation therapy immunoactive and immunoreactive agents; and a number of other agents such as transmitters and their respective receptor-agonists and -antagonists, their respective precursors or metabolites; antibiotics, antispasmodics, antihistamines, antinauseants, relaxants, stimulants, “sense” and “anti-sense” oligonucleotides, cerebral dilators, psychotropics, anti-manics, vascular dilators and constrictors, anti-hypertensives, migraine treatments, hypnotics, hyper- or hypo-glycemic agents, mineral or nutritional agents, anti-obesity drugs, anabolics and anti-asthmatics.

Still further typical active cargo molecules (e.g., therapeutic agents such as brain therapeutic agents) useful for encapsulation include any substance affecting the nervous system or used for diagnostic tests of the nervous system. These are described by Gilman et al. (1990), “Goodman and Gilman's—The Pharmacological Basis of Therapeutics”, Pergamon Press, New York, and include the following agents:
acetylcholine and synthetic choline esters, naturally occurring cholinomimetic alkaloids and their synthetic congeners, anticholinesterase agents, ganglionic stimulants, atropine, scopolamine and related antimuscarinic drugs, catecholamines and sympathomimetic drugs, such as epinephrine, norepinephrine and dopamine, adrenergic agonists, adrenergic receptor antagonists, transmitters such as GABA, glycine, glutamate, acetylcholine, dopamine, 5-hydroxytryptamine, and histamine, neuroactive peptides;
analgesics and anesthetics such as opioid analgesics and antagonists; preanesthetic and anesthetic medications such as benzodiazepines, barbiturates, antihistamines, phenothiazines and butylphenones; opioids; antiemetics; anticholinergic drugs such as atropine, scopolamine or glycopyrrolate; cocaine; chloral derivatives; ethchlorvynol; glutethimide; methyprylon; meprobamate; paraldehyde; disulfiram; morphine, fentanyl and naloxone; centrally active antitussive agents;
psychiatric drugs such as phenothiazines, thioxanthenes and other heterocyclic compounds (e.g., halperiodol); tricyclic antidepressants such as desimipramine and imipramine; atypical antidepressants (e.g., fluoxetine and trazodone), monoamine oxidase inhibitors such as isocarboxazid; lithium salts; anxiolytics such as chlordiazepoxyd and diazepam;
anti-epileptics including hydantoins, anticonvulsant barbiturates, iminostilbines (such as carbamazepine), succinimides, valproic acid, oxazolidinediones and benzodiazepines.
anti-Parkinson drugs such as L-DOPA/CARBIDOPA, apomorphine, amantadine, ergolines, selegeline, ropinorole, bromocriptine mesylate and anticholinergic agents;
antispasticity agents such as baclofen, diazepam and dantrolene;
neuroprotective agents, such as excitatory amino acid antagonists, neurotrophic factors and brain derived neurotrophic factor, ciliary neurotrophic factor, or nerve growth factor; neurotrophin (NT) 3 (NT3); NT4 and NT5; gangliosides; neuroregenerative agents;
drugs for the treatment of addiction and drug abuse include opioid antagonists and antidepressants;
autocoids and anti-inflammatory drugs such as histamine, bradykinin, kallidin and their respective agonists and antagonists;
chemotherapeutic agents for parasitic infections and microbial diseases;
anti-cancer drugs including alkylating agents (e.g., nitrosoureas) and antimetabolites; nitrogen mustards, ethylenamines and methylmelamines; alkylsulfonates; folic acid analogs; pyrimidine analogs, purine analogs, vinca alkaloids; antibiotics;
anti-nauseants, relaxants, stimulants, “sense” and “anti-sense” oligonucleotides, cerebral dilators, psychotropics, vascular dilators and constrictors, anti-hypertensives, migraine treatments, hyper- or hypo-glycemic agents, mineral or nutritional agents, anti-obesity drugs, anabolics and anti-asthmatics, anti-inflammatory drugs such as phenylbutazone, indomethacin, naproxen, ibuprofen, flurbiprofen, diclofenac, dexamethasone, prednisone and prednisolone; cerebral vasodilators such as soloctidilum, vincamine, naftidrofuryl oxalate, co-dergocrine mesylate, cyclandelate, papaverine, nicotinic acid, anti-infective agents such as erythromycin stearate, and cephalexin; and
small RNAs (like miRNAs), circular RNAs, long non-coding RNAs and immune check point inhibitors.

Pharmaceutical compositions disclosed herein include a polymer (e.g., hydrophobic polymer, e.g., a biodegradable hydrophobic polymer such as PLGA), e.g., 0.1-50 mg/mL of the hydrophobic polymer (e.g., hydrophobic polymer, e.g., a biodegradable hydrophobic polymer such as PLGA). For example, pharmaceutical compositions disclosed herein may include, e.g., 2.5 mg/mL to 50 mg/mL (e.g., 2.5-40 mg/mL, 2.5-30 mg/mL, 2.5-20 mg/mL, 2.5-10 mg/mL, 2.5-5 mg/mL, 5-50 mg/mL, 5-40 mg/mL, 5-30 mg/mL, 5-20 mg/mL, 5-10 mg/mL, 10-50 mg/mL, 10-40 mg/mL, 10-30 mg/mL, 10-20 mg/mL, 20-50 mg/mL, 20-40 mg/mL, 20-30 mg/mL, 30-50 mg/mL, 30-40 mg/mL, or 40-50 mg/mL) of a polymer (e.g., hydrophobic polymer, e.g., a biodegradable hydrophobic polymer such as PLGA). Non-limiting examples of polymers include, e.g., cyclodextrin-containing polymers, in particular cationic cyclodextrin-containing polymers, poly(caprolactone) (PCL), polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethanes, polyamino acids such as poly-L-lysine (PLL), poly(valeric acid), and poly-L-glutamic acid, hydroxypropyl methacrylate (HPMA), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), ethylene vinyl acetate polymer (EVA), polyvinyl alcohols (PVA), polyvinyl ethers, polyvinyl esters such as poly(vinyl acetate), polyvinyl halides such as poly(vinyl chloride) (PVC), and polyvinylpyrrolidone, polysiloxanes, polystyrene (PS), celluloses including derivatized celluloses such as alkyl celluloses, hydroxyalkyl celluloses, cellulose ethers, cellulose esters, nitro celluloses, hydroxypropylcellulose, and carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (jointly referred to herein as “polyacrylic acids”), polydioxanone and its copolymers, polyhydroxyalkanoates, polypropylene fumarate, polyoxymethylene, poloxamers, poly(butyric acid), trimethylene carbonate, polyphosphazenes, and combinations thereof Preferably, the polymer is PLGA. Advantageously, PLGA is biodegradable and, upon cleavage in vivo, is easily excreted from the subject's body after the therapeutic agent delivery.

The carboxyl termini of carboxylic acid contain polymers, such as lactide- and glycolide-containing polymers, may optionally be capped, e.g., by esterification, and the hydroxyl termini may optionally be capped, e.g. by etherification or esterification. Copolymers of two or more polymers described above, including block and/or random copolymers, may also be employed to make the polymeric particles. Copolymers of PEG or derivatives thereof with any of the polymers described above may be used to make the polymeric particles. In certain embodiments, the PEG or derivatives may locate in the interior positions of the copolymer. Alternatively, the PEG or derivatives may locate near or at the terminal positions of the copolymer. In certain embodiments, the microparticles or nanoparticles are formed under conditions that allow regions of PEG to phase separate or otherwise locate to the surface of the particles. The surface-localized PEG regions alone may perform the function of, or include, a surface-altering agent.

Pharmaceutical compositions disclosed herein typically include a pharmaceutically acceptable solvent, e.g., water, phosphate buffered saline (PBS), or saline.

Pharmaceutical compositions disclosed herein may be used to deliver a brain therapeutic agent to a subject in need thereof (e.g., across the blood brain barrier of the subject). Thus, disclosed are methods of delivering a brain therapeutic agent to a subject in need thereof by administration to the subject an effective amount of the pharmaceutical composition disclosed herein. The pharmaceutical composition disclosed herein may be administered, preferably, parenterally. Non-limiting examples of the routes of administration of the pharmaceutical compositions disclosed herein include, e.g., intramuscular, intravenous, intra-arterial, intracranial, subcutaneous, intraorbital, intraventricular, intraspinal, intraperitoneal, intranasal, inhalation, intradermal sublingual, buccal, transdermal, and topical administration. Preferably, the administration is intravenous. The subject may be suffering from a functional disorder (e.g., a mental disorder or a physical disorder). The functional disorder may be treated using brain therapeutic agents disclosed herein. For example, a nucleic acid (e.g., siRNA or antisense oligonucleotide) capable of suppressing the expression of tau protein may be useful in the treatment of a variety of disorders. Non-limiting examples of physical disorders that may be treated using the methods disclosed herein include, e.g., traumatic brain injury, multiple sclerosis, glioblastoma, stroke, Parkinson's disease, infectious diseases (e.g., meningitis), Alzheimer's disease, and migraine.

In one example, siRNA-loaded nanoparticles (NPs) were first fabricated by a nanoprecipitation method. 5 mg PLGA and 1 mg of lipid DSPE-PEG was dissolved in 1 ml of dimethyl formamide (DMF) solvent, and 4 nmol siRNA in 20 ul of water was mixed with the DMF solution. Next, the mixture was added into 20 ml aqueous solution containing one or more of the following components for surface modification of nanoparticles: polyethylene glycol, polysorbate 80 (0-0.2% w/v), pluronic F-68, glutathione (GSH) and transferrin. The NPs formed instantly upon mixing. NPs were washed three times in Amicon tubes (MWCO 100 kDa; Millipore) to remove remaining organic solvent and free compounds with water and resuspended in 1 mL phosphate buffered saline (PBS) solution. Their size of NPs was determined by Dynamic Light Scattering, which is about 50 nm. About 60% encapsulation efficiency was achieved for siRNA. Next, we checked if the siRNA loaded in NPs still maintain their function and could achieve gene silencing on neural cells. For these, we used luciferase siRNA as the model siRNA. The silencing experiments were performed in luciferase-expressing Neuro 2A cells. Luciferase siRNA-loaded NPs showed significant silencing of the luciferase expression. Nearly 90% luciferase silence was obtained with 10-20 nM siRNA. No obvious cytotoxicity was observed under these conditions. The internalization of siRNA-loaded NPs into neuro cells was evaluated by confocal microscopy. siRNA loaded nanoparticles showed significantly higher cellular uptake over free siRNA. And demonstrated efficient endosomal escape after 2 hours of incubation with cells at 37° C.

In another example, the effect of different surface coatings on siRNA loaded PLGA nanoparticles on brain accumulation across intact blood brain barrier in healthy mice was studied. Mice were intravenously injected with Cy 5.5 labeled siRNA loaded nanoparticles coated with only PEG or PEG with polysorbate 80, GSH or transferrin. Following 4 h and 24 h of injection, animals were sacrificed, and brains were imaged using in vivo imaging system (IVIS).

Addition of Polysorbate 80 and GSH in surface coating showed higher improvement in brain accumulation of nanoparticles compared to only PEG coating or PEG+transferrin coating. We also observed that brain accumulation of these NPs depends upon density of surface coating on nanoparticle. Specifically, NPs with high surface coating densities of Poly80 showed remarkably higher brain accumulation compared to low and medium coating density.

Next, the siRNA NP platform was shown to silence a potential therapeutic target in traumatic brain injury (TBI). Tau protein is highly involved in brain injury, and it has been reported as an efficient target in TBI treatment. Immunofluorescence staining illustrated that Tau siRNA-loaded NPs successfully knocked down the expression of Tau in primary neural cells isolated from mouse embryo. The western blot analysis also showed that NPs dramatically reduced Tau protein levels of primary neuro cells.

To demonstrate brain accumulation of Tau siRNA loaded nanoparticles in vivo, the near infrared dye DY677-labeled siRNA was used. We used the standard weight drop model of TBI. DY677 siRNA-loaded were injected into TBI mice, either immediately after the injury or 2 weeks after the injury. Mice that received siRNA-NPs injections show significantly higher brain signal than free siRNA-injected mice. In addition, even after 2 weeks, when the blood-brain-barrier (BBB) is repaired, the NPs were still able to enter the brain. The sections of the nanoparticle-injected mice brains were also studied by confocal microscopy to observe the siRNA signal in brain. Accumulation of siRNA in the brain was found in the siRNA NPs-injected mice but not from the free siRNA injected mice. And the siRNA signal was widely distributed in the brain, not localized in the blood vessel, which showed the NP entered the brain tissue.

Next, we tested if the Tau-siRNA-loaded NPs could achieve in vivo Tau silencing in TBI mice. TBI injury was induced on day 0 followed by a tail vein injection of free siRNA, or siRNA loaded nanoparticles. Brains were harvested on day 4 for Western blot analysis of Tau expression. Tau siRNA-loaded NPs led to around 40-50% of knockdown of Tau expression in the brain tissue of TBI mice. The free siRNA on the other hand did not show any effect. We further studied if we could achieve Tau silencing by administering NPs at late injury phase, when the blood brain barrier is repaired. siRNA loaded NPs could still efficiently silence Tau expression.

Features of the present disclosure include the following.
1. Nanoparticles with surface coating that improves their brain accumulation across both intact and physically breached blood brain barrier.
2. Ability to deliver encapsulated agents, including both hydrophobic and hydrophilic drugs and biologics such as siRNA across blood brain barrier upon, e.g., intravenous administration.
3. Nanoparticles demonstrate gene silencing in brain in a traumatic brain injury model.

The following examples further illustrate the invention. They are not meant to limit the invention in any way.

### EXAMPLES

### Example 1: Influence of Surface Coating on the Ability of NPs for Brain siRNA Delivery

To investigate how the surface coating influences the ability of NPs for brain siRNA delivery, five PLGA NP formulations with different surface chemistries were designed, including the (1) PEG-coated siRNA-loaded NPs, (2) Poloxamer 188 (F 68)-coated siRNA-loaded NPs, (3) Polysorbate 80 (PS 80)-coated siRNA-loaded NPs, (4) glutathione (GSH)-coated siRNA-loaded NPs, and (5) transferrin (Tf)-coated siRNA-loaded NPs.

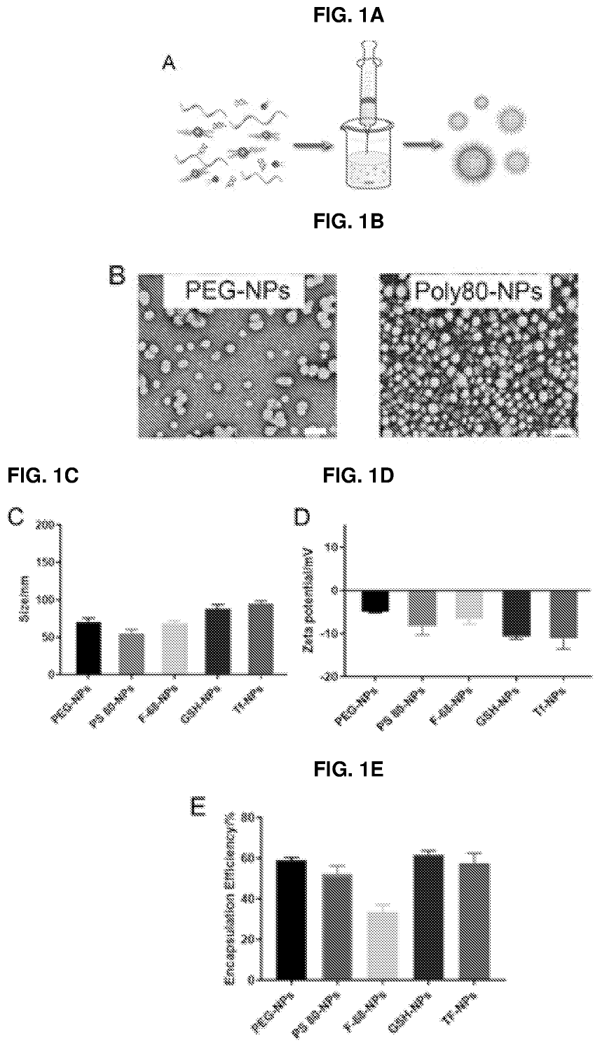

A modified nanoprecipitation approach was employed to fabricate the siRNA-loaded PLGA NP formulations (FIG. 1A). 5 mg PLGA, 1 mg cationic lipid, and 4 nmol siRNA were dissolved in 1 ml DMF to form a homogenous solution. The organic mixture was added slowly into aqueous solution. The various coating materials were added into either the organic phase or water phase to make the PLGA NPs with different coating, as shown in the table below.

| | Organic phase containing | Water phase containing |
|---|---|---|
| PEG PLGA-NPs | 2.5 mg/ml DSPE-PEG | water |
| Tf PLGA-NPs | 0.5 mg/ml DSPE-PEG-Tf + | water |
| | 2.0 mg/ml DSPE-PEG | |
| GSH (L) PLGA-NPs | 0.25 mg/ml DSPE-PEG-GSH + | water |
| | 2.25 mg/ml DSPE-PEG | |
| GSH (M) PLGA-NPs | 0.5 mg/ml DSPE-PEG-GSH + | water |
| | 2.0 mg/ml DSPE-PEG | |
| GSH (H) PLGA-NPs | 1.25 mg/ml DSPE-PEG-GSH + | water |
| | 1.25 mg/ml DSPE-PEG | |
| F68 PLGA-NPs | 1.25 mg/ml DSPE-PEG | 1 mg/ml F 68 |
| PS 80 (L) PLGA-NPs | 0.5 mg/ml DSPE-PEG | 0.4 mg/ml PS80 |
| PS 80 (M) PLGA-NPs | 1.25 mg/ml DSPE-PEG | 1 mg/ml PS80 |
| PS 80 (MH) PLGA-NPs | 1.875 mg/ml DSPE-PEG | 1.5 mg/ml PS80 |
| PS 80 (H) PLGA-NPs | | 2 mg/ml PS80 |

The physiochemical properties of the five NP formulations were then characterized. They exhibited a spherical morphology with an average diameter of 40-70 nm, when observed by transmission electron microscope (TEM) image (FIG. 1B) and a hydrodynamic diameter of 55-95 nm when measured by DLS analysis (FIG. 10). Results analyzing zeta potential showed that all the NPs had a slightly negative surface charge. Using Cy3-labelled siRNA as an indicator, the encapsulation efficiencies of siRNA in different coating-modified PLGA NPs were found to be approximately 60%, while only the encapsulation efficiency of 35% was achieved for the F-68-coated NPs. Due to the low siRNA loading ability, the F-68 NP formulation was no longer used in the following comparison.

### Example 2: Internalization of Different siRNA-Loaded Nanoplatforms by Neuro-2a Cells

The internalization of different siRNA-loaded nanoplatforms by Neuro-2a cells was studied. siRNA was labeled with a red fluorescence probe, and the signal of siRNA-loaded NPs in cells was evaluated qualitatively via confocal laser scanning microscope (CLSM).

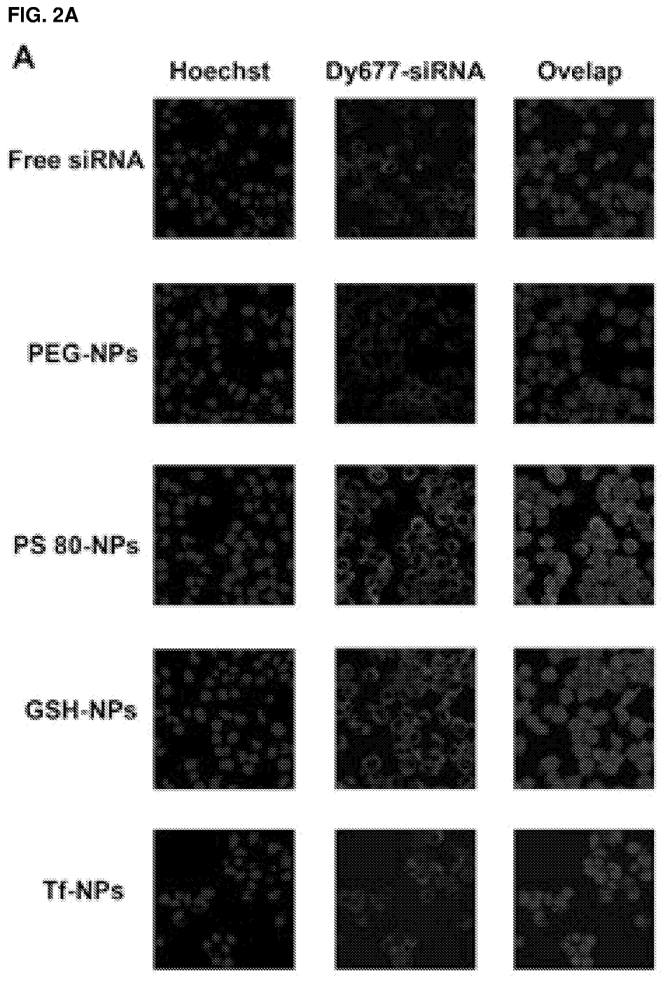

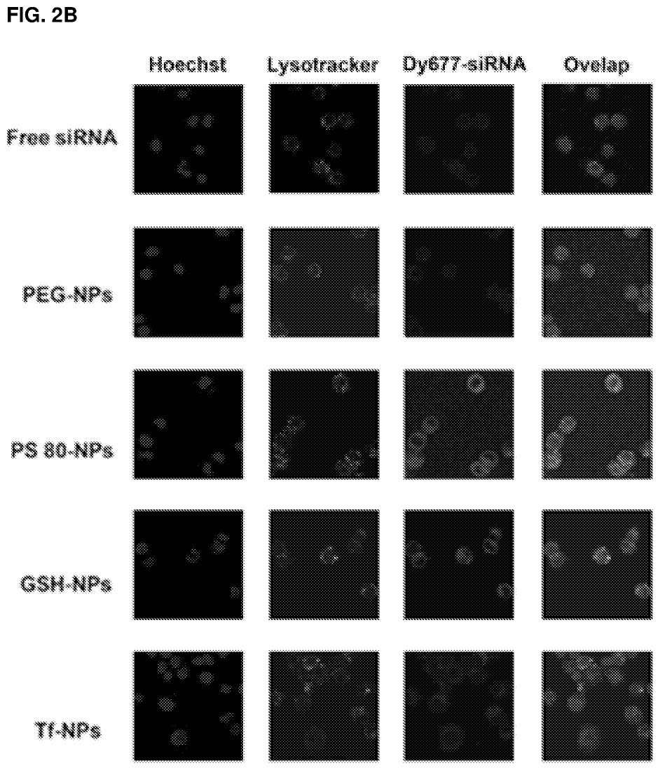

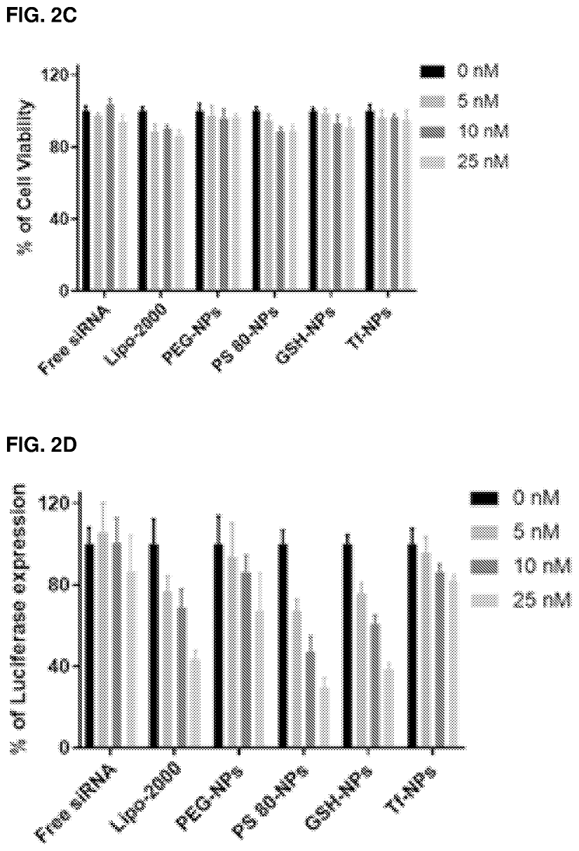

Weak fluorescence was observed for the PEG-coated NP formulation, demonstrating that the PEG coating reduced the interaction between NPs and cells (FIG. 2A). The incorporation of Tf or GSH onto PLGA NPs enhanced the cellular uptake of the nanocarriers. The siRNA-loaded NPs presenting PS 80 showed higher cellular uptake as observed by intense fluorescence signal inside cells.

After entering cells, siRNA must escape from the endosomes to engage the cytoplasmic RNAi machinery for gene silencing. The endosomal escape of siRNA was also assessed by using green Lysotracker to label endosomes of cells. The distribution of red dye-labeled siRNA inside cells was studied by CLSM. It was found that a majority of the internalized siRNA-loaded NPs left the lysosomes, spreading into the cytoplasm of cells after 2 hours incubation (FIG. 2B).

To assess the effect of NP surface chemistries on gene silencing, luciferase siRNA was encapsulated into different NP formulations and a luciferase-expressing Neuro-2A cell line was engineered by transduction of cells with luciferase expression vector. First, we studied the cytotoxicity of NPs. No notable reduction in cell viability was observed with more than 90% NP-treated cells maintaining alive (FIG. 2C).

Gene silencing efficacy was also examined (FIG. 2D). All the siRNA-loaded NPs suppressed the luciferase expression in a dose-dependent manner. However, the silencing efficacy of NPs was found to vary with the surface coating: PS 80- and GSH-coated NPs demonstrated more potent silencing than the PEG- and Tf-coated NPs. In particular, the NPs presenting PS 80 exhibited highest knockdown in luciferase expression.

### Example 3: Efficacy of Surface Chemistries for Delivering Therapeutic Agents In Vivo to the Brain

siRNA delivery efficacy of different surface chemistries-coated NPs in vivo was studied as follows. For this purpose, the near infrared dye DY677-tagged siRNA was loaded into various NP platforms. The naked siRNA and siRNA-loaded NPs were intravenously injected to healthy mice via tail vein. The mice brains were harvested and imaged by in vivo imaging systems (IVIS).

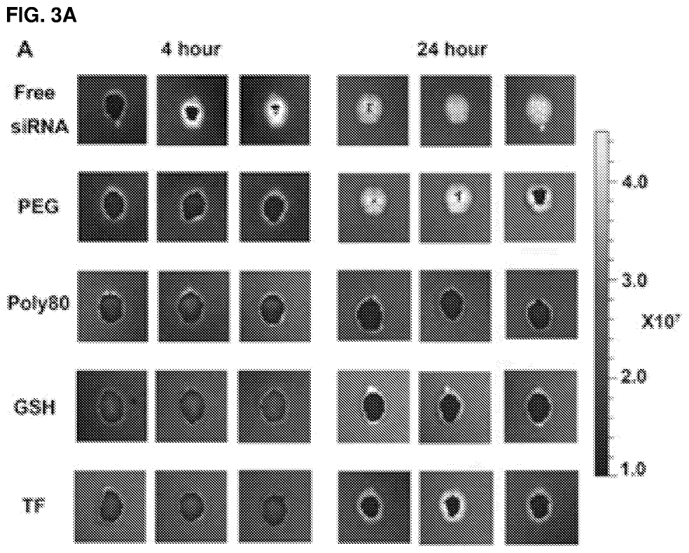

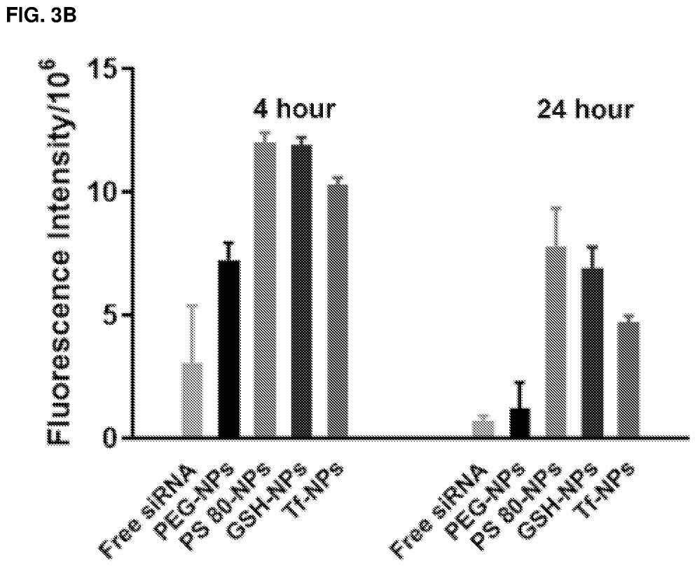

As shown in the images and quantification analysis (FIGS. 3A and 3B), the naked siRNA exhibited negligible signal in brain. In contrast, high accumulations of the PS-80 coated and GSH-coated NPs in brain were observed. The signal of PEG- and Tf-coated NPs in brain was found to be lower than that of PS 80- or GSH-coated NPs. Collectively, these results confirmed that the surface chemistries influence the in vitro and in vivo performance of NPs.

As the PS- and GSH-coated NPs showed both higher gene silencing and more effective brain accumulation than the other coatings-modified nanoplatforms, these were chosen for further modifications.

### Example 4: Effects of PS 80 and GSH Coating Density on the Performance of NPs

The effects of coating density on the performance of NPs were analyzed as follows. For this purpose, the siRNA-loaded PLGA NPs with different coating densities of PS 80 and GSH were prepared.

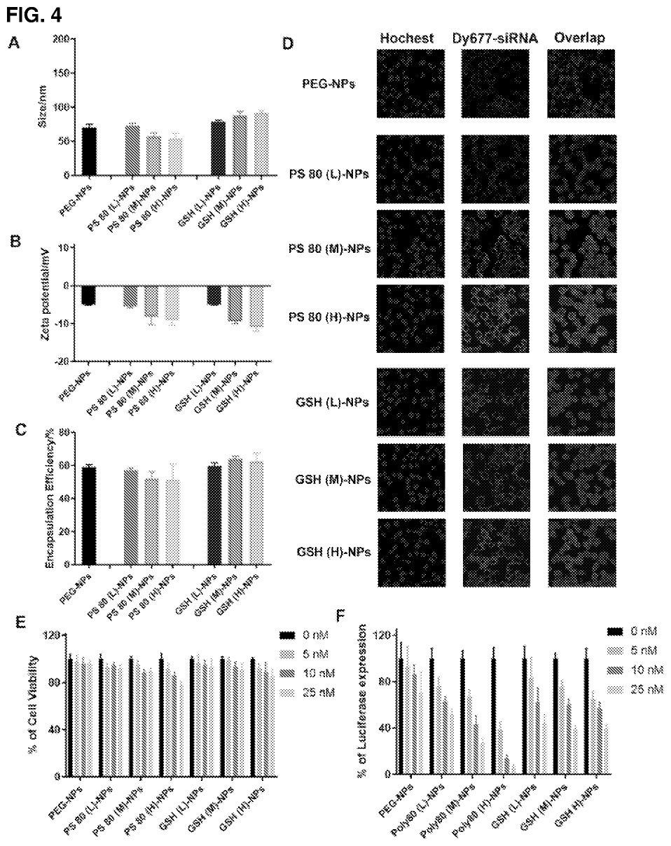

The NPs with low, medium, and high coating density of PS 80 and GSH were here designated as PS 80 (L) NPs, PS 80 (M) NPs, PS 80 (H) NPs, GSH (L) NPs, GSH (M) NPs, and GSH (H) NPs respectively. The NPs with low, medium, medium-high, or high coating density of PS 80 were prepared by adding the organic mixture into the aqueous solution containing 0.4 mg/ml, 1 mg/ml, 1.5 mg/ml or 2 mg/ml of PS80, and the obtained NPs were designated as PS 80 (L) NPs, PS 80 (M) NPs, PS 80 (MH) NPs and PS 80 (H) NPs respectively. The NPs with low, medium, or high coating densities of GSH were prepared by adding the organic mixture containing 0.25 mg/ml, 0.5 mg/ml, or 1.25 mg/ml of DSPE-PEG-GSH into the aqueous solution, and the obtained NPs were designated as GSH (L) NPs, GSH (M) NPs, and GSH (H) NPs respectively. The DLS analysis implied that the sizes of these NPs were all in the range of 50-80 nm (FIG. 4A). The zeta potential of NPs became more negative when increasing the surface coating densities of PS 80 and GHS (FIG. 4B). The coating density did not noticeably change the siRNA loading ability of NPs with the siRNA encapsulation efficiency around 50-60% (FIG. 4C).

The in vitro cellular uptake and gene silencing ability of these NPs was also examined. High-level cellular uptake was observed for NPs prepared with high density of PS 80.

In addition, the gene silencing efficacy of these different NPs in neural cells was also examined (FIG. 4F). Among the nanoplatforms, the NPs with high density of PS 80 displayed most effective gene silencing. Notably, there was no obvious cytotoxicity of the NPs used for these in vitro transfection experiment (FIG. 4E).

### Example 5: In Vivo Brain Accumulation of PS 80 and GSH Coated NPs

After comparing the cellular uptake and gene silencing ability of NPs with different coating densities of PS 80 and GSH, their in vivo brain accumulation behavior was analyzed as follows.

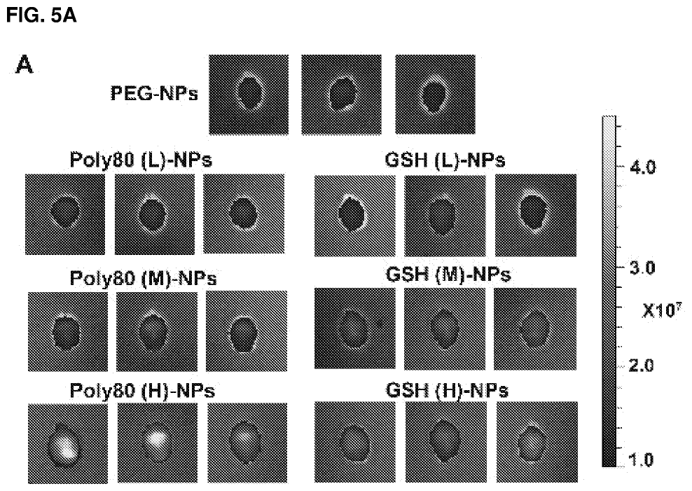

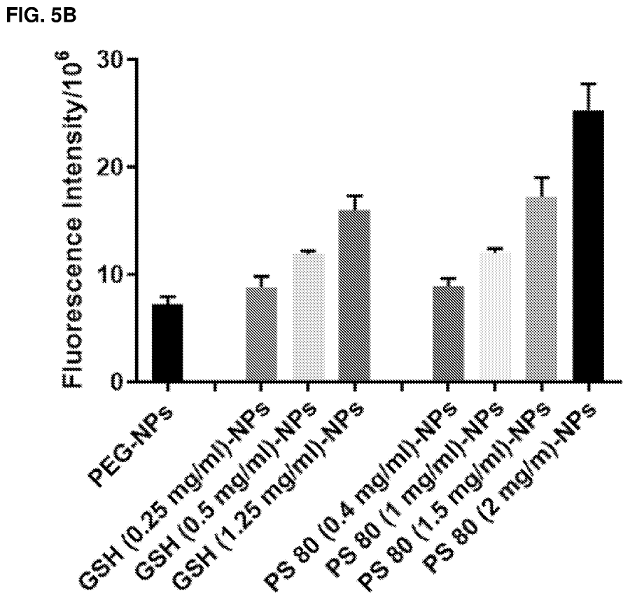

After intravenous injection of different DY677-siRNA-loaded NPs into healthy mice, the brains of mice were collected for IVIS imaging. As shown in the images in FIG. 5A and quantified analysis in FIG. 5B, the brains of mice injected with P80 (H)-coated PLGA NPs exhibited strong fluorescence, which was 4 times higher than that of the PEG-coated NPs-treated mice brain. Although the GSH coating also enhanced the transport of NPs into brain when compared with PEG coating, the levels of GSH (H)-NPs in brain were lower than that of PS 80(H)-NPs.

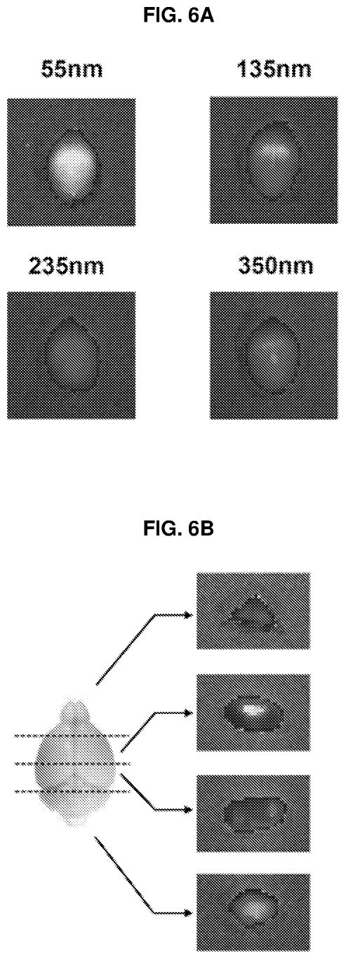

Next the effect of NPs' size on brain accumulation was tested. By changing the organic solvent and the initial polymer's concentration used for nanoparticles synthesis, the siRNA-loaded PS 80 NPs with different size were gained. Here, the NPs with 4 different size, 55 nm, 135 nm, 235 nm, and 350 nm were tested. The siRNA encapsulation efficiency of these NPs was between 40-65%. These NPs were injected into mice and the brains were collected after 4 hours. As shown in the IVIS images (FIG. 6A), the small one exhibited higher brain accumulation than large ones. For further study, the small sized PS80 NPs were chosen. To show the distribution of siRNA signal in brain, the brain tissue was divided into four different parts with coronal sections, and they were imaged with IVIS. As shown in FIG. 6B, we see higher siRNA signal in the middle center of brain.

### Example 6: PS 80 (H) NP for Treatment of TBI

After comparing the various surface-coated PLGA NP formulations, the PS 80 (H) NP was chosen as the carrier to deliver siRNA for TBI treatment due to its optimal performance in gene silencing and brain accumulation. The ability of PS 80 (H) NPs in mitigating the destructive pathways involved in TBI and improving the functional outcome of TBI mice were then assessed. Among various destructive pathways, Tau protein pathology was found to be highly correlated with the chronic neuroinflammation, neurodegenerative process, and cognitive impairment caused by TBI. Thus, we want to test if the optimized nanoplatform PS 80 (H) NPs could provide a noninvasive method to deliver Tau siRNA into TBI mice and show any therapeutic effect.

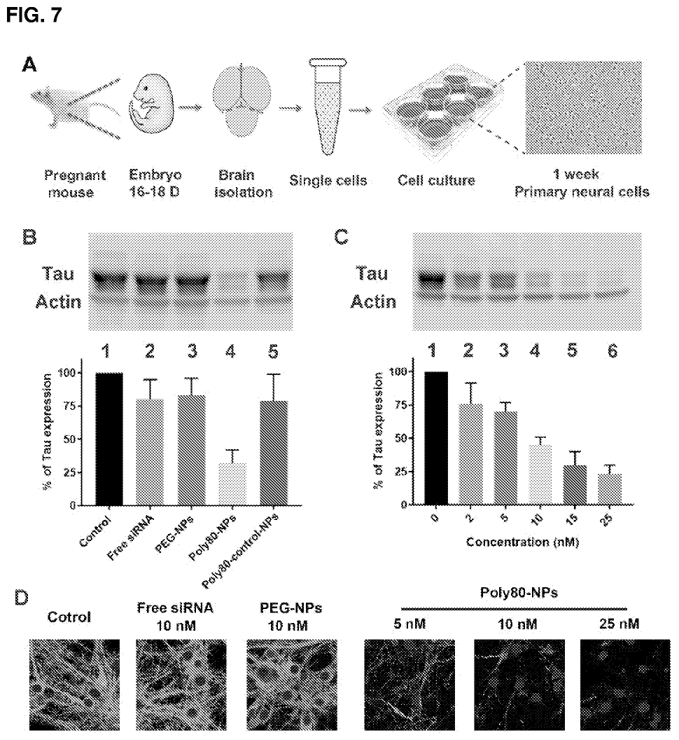

To study the in vitro Tau silencing ability of PS 80 (H) NPs in neural cells, we isolated the primary mouse hippocampal and cortical neurons from embryonic mice. Next, we examined whether Tau siRNA-loaded PS 80 (H) NPs could downregulate Tau expression in primary neuron cells. The western blot analysis in FIG. 7B indicated that Tau siRNA-loaded PS 80 (H) NPs could significantly suppress the Tau expression in primary neurons (FIG. 7B). The Tau siRNA-loaded PS 80 (H) NPs showed a dose-dependent knockdown with more significant Tau silencing observed at higher siRNA doses (FIG. 7C). The similar results were found in the immunostaining analysis (FIG. 7D). Compared to cells treated with free Tau siRNA or Tau siRNA-loaded PEG NPs, the cells treated with Tau siRNA-loaded PS 80 (H) NPs displayed much weaker green fluorescence corresponding to the lower expression level of Tau protein.

### Example 7: Performance of PS 80 (H) NPs In Vivo in TBI Mice by Using the Weight-Drop TBI Model

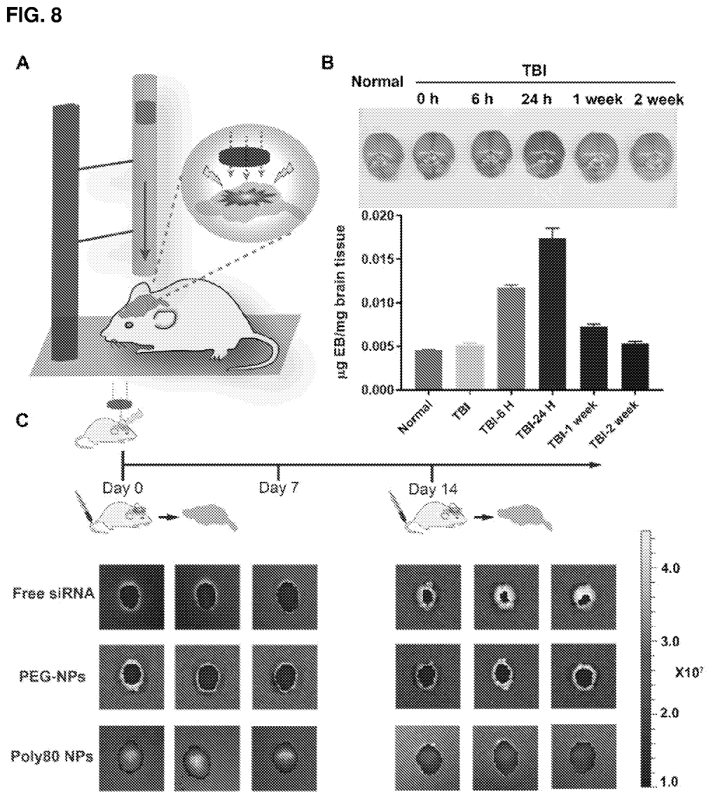

After validating the efficient gene silencing of PS 80 (H) NPs, we then evaluated their in vivo performance in TBI mice by using the weight-drop TBI model (FIG. 8A). The BBB permeability following the weight-drop method-induced TBI was characterized by Evans blue (EB) penetration assay. After injury, the level of EB extravasation in mice brain tissue was gradually elevated. Compared to sham animals, the brain tissue of TBI mice displayed a blue color and higher EB uptake at 24 hours post-injury. The closure of BBB to EB permeability was observed 7 days after injury, which suggested that the BBB of TBI mice was nearly self-repaired after 1 week (FIG. 8B).

The DY677 siRNA, DY677 siRNA-loaded PEG NPs or PS 80 (H) NPs were then administered into TBI mice via intravenous tail vein injection. Low levels of free siRNA or siRNA-loaded PEG NPs accumulated into the mice brain with weak fluorescence signal detected in the isolated brain tissues. However, for the siRNA-PS 80 (H) NPs, we observed a strong fluorescent signal in the brain (FIG. 8C). To evaluate the siRNA delivery in late injury period, the free siRNA or siRNA-loaded NPs were also administrated intravenously 2 weeks after injury (FIG. 8D). Among the different formulation-injected mice, the mice treated with siRNA-PS 80 (H) NPs again displayed the highest levels of siRNA fluorescence signal in brain. These results implied that the PS 80 (H) NPs were able to efficiently deliver siRNA into brain both within and outside the transient BBB disruption window, which will show great potential for downregulating pathogenic targets involved in secondary brain injury, a process that usually lasts weeks or months.

### Example 8: In Vivo Tau Silencing

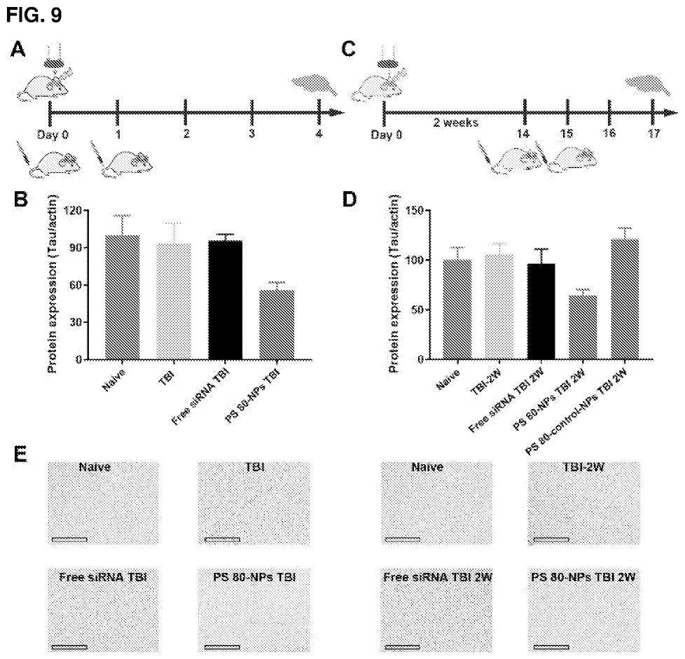

We next advanced the nanoplatforms into in vivo tau silencing study. The free Tau siRNA, and PS 80 NPs carrying either control siRNA or siRNA against Tau were injected intravenously into TBI mice for 2 days. At four days post-injury, the brains were isolated for analysis of Tau expression by using western blotting (FIGS. 9A and 9B). We did not observe any Tau knockdown after free siRNA treatment, which was in agreement with the limit delivery of free siRNA to brain. When Tau siRNA-loaded PS 80 NPs were injected, obvious decrease of Tau protein level in mice brain was achieved. The control siRNA-loaded PS 80 NPs, however, showed negligible Tau silencing ability, which indicated that the PS 80 NPs itself has little effect on Tau knockdown. Interestingly, even administered 2 weeks after the injury, the Tau siRNA-loaded PS 80 NPs could still block Tau expression in brain tissue (FIGS. 9C and 9D). A similar tendency was found in the immunohistochemical staining results. As shown in FIG. 9E, the Tau protein level was dramatically reduced in the brain from mice subjected to Tau siRNA-loaded PS 80 NPs, but not from the free siRNA-treated mice.

### Example 9: In Vitro Nanoparticle Permeability Assay

bEnd.3 cells (ATCC) were grown on 1% gelatin-coated flasks at 37° C., 5% CO2 in DMEM with 10% FBS and 1% penicillin/streptomycin (10,000 U/mL). Then, the cells were seeded on 2% growth factor reduced Matrigel-coated 12 mm Transwell® with 0.4 μm pore polycarbonate membrane insert (Corning) at a density of 80,000 cells/well. Media was replaced every two/three days. Transendothelial electrical resistance (TEER) was measured using an EVOM resistance meter (World Precision Instruments). Once TEER had reached ≥50 ohm*cm² (after 1 week), permeability experiments were performed. Labelled nanoparticles were added to the apical compartment in serum-free DMEM. After 4 h, filter inserts were withdrawn from the receiver compartment. Aliquots from the apical and basolateral compartments were collected and the fluorescence was quantified using Infinite Pro 200 plate reader from Tecan. At least three inserts with cells were used in each permeability measurement. Empty filters without cells were used to determine the maximum nanoparticle permeability in vitro. Mouse transferrin (Tf; Sigma) and low density lipoprotein receptor related protein 1 (LRP1) antibody (CST) were used to block receptor-mediated uptake of nanoparticles by bEnd.3 cells. Both compartments of the transwell incubated with different concentrations of mTf (1 and 2 mg/mL) or LRP1 antibody (100 and 200 μM) in DMEM prior to nanoparticle introduction. After 1 h, the particles were introduced to the apical compartment in the presence of mTf or LRP1 antibody. The assay was performed as described above. Three wells were used for each nanoparticle formulation in these assays.

To predict the passage of nanoparticles through BBB, an in vitro BBB monoculture model was established using mouse bEnd.3 cell line. Labelled nanoparticles were added to the apical part of the bEnd.3-containing transwells in serum-free DMEM and allowed to cross the endothelial cell layer for 4 h. Empty filters without cells were used to determine the maximum passage of nanoparticles in the transwell system and considered as the condition with %100 permeability. After 4 h, medium samples were collected from apical and basolateral parts and fluorescence measurements were done.

Here six PLGA NP formulations with different surface chemistries were tested for the in vitro BBB model, including the PEG PLGA-NPs, GSH PLGA-NPs, Tf PLGA-NPs, PS80 PLGA-NPs, F127 PLGA-NPs, and TPGS PLGA-NPs. For GSH PLGA-NPs and Tf PLGA-NPs were prepared by adding the organic mixture containing 1.25 mg/ml of DSPE-PEG-GSH or 0.5 mg/ml DSPE-PEG-Tf into the aqueous solution respectively. And PS80 PLGA-NPs, F127 PLGA-NPs, and TPGS PLGA-NPs were prepared by adding the organic mixture into the aqueous solution containing 2 mg/ml of PS80, 3 mg/ml F127, or 2 mg/ml TPGS respectively.

According to the results, TPGS, PS80, F127 and Tf nanoparticle groups showed the highest capacity of crossing BBB in vitro. TPGS and PS80 nanoparticles showed around 4 and 3 times more nanoparticle passage, respectively, compared with the widely used PEG nanoparticles.

Certain macromolecules can reach the brain via receptor-mediated transcytosis (RMT) and BBB has several receptors such as insulin receptor, transferrin receptor, and receptors responsible for lipoprotein transport. Targeting these receptors could facilitate the delivery of drugs/nanoparticles to the brain. Transferrin receptor and low-density lipoprotein receptor-related protein 1 (LRP1) are two main receptors for RMT. We checked whether these receptors could play a role in the higher permeability of PS80 nanoparticles through BBB. For this purpose, bEnd.3 cells cultured on transwell inserts incubated with different concentrations of mTf (1 and 2 mg/mL) or LRP1 antibody (100 and 200 μM) in DMEM 1 h prior to nanoparticle introduction. After 1 h, the particles were introduced to the apical compartment in the presence of mTf or LRP1 antibody. The results showed that the passage of PS80 nanoparticles were mediated by LRP1 and not by Tf receptor. Tf nanoparticles were also used as controls in these experiments. The permeability of these nanoparticles was reduced by the addition transferrin as expected.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A pharmaceutical composition formulated for delivery to the brain of a subject, the composition comprising a plurality of nanoparticles (NPs) comprising a brain therapeutic agent, poly(lactic-co-glycolic acid) (PLGA), and a pharmaceutically acceptable excipient selected from the group consisting of a surfactant, peptide, and combinations thereof.

2. The pharmaceutical composition of claim 1, wherein the brain therapeutic agent treats a functional disorder.

3. The pharmaceutical composition of claim 1, wherein the brain therapeutic agent treats a physical disorder.

4. The pharmaceutical composition of claim 3, wherein the physical disorder is a traumatic brain injury.

5. The pharmaceutical composition of claim 1, wherein the pharmaceutical composition comprises a surfactant.

6. The pharmaceutical composition of claim 5, wherein the surfactant is a polysorbate, polyethylene glycol, or poloxamer.

7. The pharmaceutical composition of claim 6, wherein the surfactant is a polysorbate.

8. The pharmaceutical composition of claim 7, wherein the polysorbate is polysorbate 80.

9. The pharmaceutical composition of claim 1, wherein the pharmaceutical composition comprises 0.001-0.2% (w/v) of the surfactant.

10. The pharmaceutical composition of claim 1, wherein the pharmaceutical composition comprises 0.1-0.2% (w/v) of the surfactant.

12. The pharmaceutical composition of claim 1, wherein the pharmaceutical composition comprises a peptide.

13. The pharmaceutical composition of claim 11, wherein the peptide is glutathione, transferrin, or a combination thereof.

14. The pharmaceutical composition of claim 12, wherein the peptide is glutathione.

15. The pharmaceutical composition of claim 1, wherein the pharmaceutical composition comprises 0.05-0.5% (w/v) of the peptide.

16. The pharmaceutical composition of claim 1, wherein the pharmaceutical composition comprises 0.1-0.2% (w/v) of the peptide.

17. The pharmaceutical composition of claim 1, wherein the NPs have an average hydrodynamic diameter of 40-150 nm, as measured by Dynamic Light Scattering.

18. The pharmaceutical composition of claim 1, wherein the NPs have an average hydrodynamic diameter of 40-100 nm, as measured by Dynamic Light Scattering.

19. The pharmaceutical composition of claim 1, wherein the NPs have an average hydrodynamic diameter of 55-95 nm, as measured by Dynamic Light Scattering.

20. The pharmaceutical composition of claim 1, wherein the average molecular weight of the NPs is 7-31 kDa.

21. The pharmaceutical composition of claim 1, wherein the average diameter of the NPs is 40-70 nm, as measured by transmission electron microscopy (TEM).

22. The pharmaceutical composition of claim 1, wherein the brain therapeutic agent is a nucleic acid.

23. The pharmaceutical composition of claim 22, wherein the nucleic acid is a plasmid, siRNA, shRNA, miRNA, antisense oligonucleotide, gRNA, aptamer, or a combination thereof.

24. The pharmaceutical composition of claim 23, wherein the nucleic acid is siRNA or antisense oligonucleotide.

25. The pharmaceutical composition of claim 1, wherein the brain therapeutic agent is selected from the group consisting of anti-inflammatory drugs, steroids, antibiotics, immunosuppressants, chemotherapeutics, sensitizing agents, antibodies, antibody fragments, proteins, peptides, growth factors, cytokines, cells, stem cells, vitamins, and combinations thereof.

26. The pharmaceutical composition of claim 1, wherein the pharmaceutical composition comprises a solvent.

27. The pharmaceutical composition of claim 23, wherein the solvent is water, saline, or phosphate-buffered saline.

28. The pharmaceutical composition of claim 1, wherein the pharmaceutical composition comprises 0.5-50 mg/mL of PLGA.

29. A method of delivering a brain therapeutic agent to a subject in need thereof, the method comprising administering to the subject an effective amount of the pharmaceutical composition of claim 1.

30. The method of claim 29, wherein the method treats a functional disorder in the subject,

31. The method of claim 30, wherein the functional disorder is a mental disorder.

32. The method of claim 30, wherein the functional disorder is a physical disorder.

33. The method of claim 32, wherein the physical disorder is a traumatic brain injury.

34. The method of claim 30, wherein the functional disorder is a traumatic brain injury and the brain therapeutic agent is a nucleic acid.

35. The method of claim 29, wherein the pharmaceutical composition is administered intravenously.

36. A pharmaceutical composition comprising a plurality of nanoparticles (NPs) comprising a cargo molecule, a hydrophobic polymer, and a pharmaceutically acceptable excipient selected from the group consisting of a surfactant, peptide, and combinations thereof.

37. The pharmaceutical composition of claim 36, wherein the pharmaceutical composition comprises a surfactant.

38. The pharmaceutical composition of claim 36, wherein the surfactant is a polysorbate, polyethylene glycol, or poloxamer.

39. The pharmaceutical composition of claim 38, wherein the surfactant is a polysorbate.

40. The pharmaceutical composition of claim 39, wherein the polysorbate is polysorbate 80.

41. The pharmaceutical composition of claim 36, wherein the pharmaceutical composition comprises 0.001-0.2% (w/v) of the surfactant.

42. The pharmaceutical composition of claim 36, wherein the pharmaceutical composition comprises 0.1-0.2% (w/v) of the surfactant.

43. The pharmaceutical composition of claim 36, wherein the pharmaceutical composition comprises a peptide.

44. The pharmaceutical composition of claim 36, wherein the peptide is glutathione, transferrin, or a combination thereof.

45. The pharmaceutical composition of claim 44, wherein the peptide is glutathione.

46. The pharmaceutical composition of claim 36, wherein the pharmaceutical composition comprises 0.05-0.5% (w/v) of the peptide.

47. The pharmaceutical composition of claim 36, wherein the pharmaceutical composition comprises 0.1-0.2% (w/v) of the peptide.

48. The pharmaceutical composition of claim 36, wherein the NPs have an average hydrodynamic diameter of 40-150 nm, as measured by Dynamic Light Scattering.

49. The pharmaceutical composition of claim 36, wherein the NPs have an average hydrodynamic diameter of 40-100 nm, as measured by Dynamic Light Scattering.

50. The pharmaceutical composition of claim 36, wherein the NPs have an average hydrodynamic diameter of 55-95 nm, as measured by Dynamic Light Scattering.

51. The pharmaceutical composition of claim 36, wherein the average molecular weight of the NPs is 7-31 kDa.

52. The pharmaceutical composition of claim 36, wherein the average diameter of the NPs is 40-70 nm, as measured by transmission electron microscopy (TEM).

53. The pharmaceutical composition of claim 36, wherein the cargo molecule is a nucleic acid, an anti-inflammatory drug, a steroid, an antibiotic, an immunosuppressant, a chemotherapeutic, a sensitizing agent, an antibody or fragment thereof, a protein or peptide, a growth factor, a cytokine, a cell, a vitamin, or any combination of the foregoing cargoes.

54. The pharmaceutical composition of claim 36, wherein the pharmaceutical composition comprises a solvent.

55. The pharmaceutical composition of claim 54, wherein the solvent is water, saline, or phosphate-buffered saline.

56. The pharmaceutical composition of claim 36, wherein the hydrophobic polymer is a biodegradable hydrophobic polymer.

57. The pharmaceutical composition of claim 36, wherein the hydrophobic polymer is poly(lactic-co-glycolic acid) (PLGA), poly(caprolactone) (PCL), poly(lactic acid) (PLA), poly(glycolic acid) (PGA), poly(caprolactone)-co-poly(lactic acid) (PCLLA), poly(L-lactic acid) (PLLA), or poly(glycerol sebacate) acrylate (PGSA).

58. The pharmaceutical composition of claim 36, wherein the hydrophobic polymer is PLGA.

59. The pharmaceutical composition of claim 36, wherein the pharmaceutical composition comprises 0.5-50 mg/mL of the hydrophobic polymer.
